SEQUENCE LISTING (19 sequences)

SEQ ID NO: 1 - protein, 93 aa, Homo sapiens
  MISC_FEATURE: Human S100A8/Mrp8 protein sequence
MLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 2 - protein, 114 aa, Homo sapiens
  MISC_FEATURE: Human S100A9/Mrp14 protein sequence
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 3 - protein, 230 aa, Artificial
  Sequence of HisTag-3C Protease Seq.-MRP14-3C Protease Seq.[Linker]-MRP8
MHHHHHHLEVLFQGPMTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTPLEVLFQGPMLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 4 - protein, 229 aa, Artificial
  MRP8-3C Protease Seq.[Linker]-MRP14-3C Protease Seq.-HisTag
MLTELEKALNSIIDVYHKYSLIKGNFHAVYRDDLKKLLETECPQYIRKKGADVWFKELDINTDGAVNFQEFLILVIKMGVAAHKKSHEESHKELEVLFQGPMTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTPLEVLFQGPHHHHHH

SEQ ID NO: 5 - DNA, 693 bp, Artificial
  DNA sequence of HisTag-3C Protease Seq.-MRP14-3C Protease Seq.[Linker]-MRP8 for expression in E.coli
atgcaccatcatcaccatcacttggaagttctgtttcaaggcccaatgacgtgcaaaatgagccagttggaacgcaatattgaaaccatcattaacacctttcatcagtattccgtcaaactgggtcatccggatacccttaatcaaggggaatttaaagagctggtacgcaaagatctccagaatttcctgaagaaagaaaacaagaacgagaaagtcatcgaacacattatggaagatctggataccaacgcggataaacagttatcgtttgaggagttcattatgctgatggcacgtctgacatgggcctcacatgagaaaatgcatgaaggcgatgagggtcctggacatcatcacaaaccggggttaggcgaaggaactccgttagaagtgttgtttcaaggtccgatgctgacggaactggagaaagcgcttaacagcattattgacgtttaccacaaatacagtctgatcaaaggcaatttccatgcggtatatcgtgacgatctgaagaaacttctcgaaacggaatgtccccagtatatccgcaagaaaggtgccgatgtgtggttcaaagaactggacattaacactgacggtgcagtgaatttccaggaatttctcattctggtgatcaaaatgggcgttgctgctcacaagaaatcgcatgaagagtctcacaaagaataa

SEQ ID NO: 6 - DNA, 690 bp, Artificial
  DNA sequence for expression of MRP8-3C Protease Seq.[Linker]-MRP14-3C Protease Seq.-HisTag in E.coli
atgctgacggaactggagaaagcgcttaacagcattattgacgtttaccacaaatacagtctgatcaaaggcaatttccatgcggtatatcgtgacgatctgaagaaacttctcgaaacggaatgtccccagtatatccgcaagaaaggtgccgatgtgtggttcaaagaactggacattaacactgacggtgcagtgaatttccaggaatttctcattctggtgatcaaaatgggcgttgctgctcacaagaaatcgcatgaagagtctcacaaagaattggaagttctgtttcaaggcccaatgacgtgcaaaatgagccagttggaacgcaatattgaaaccatcattaacacctttcatcagtattccgtcaaactgggtcatccggatacccttaatcaaggggaatttaaagagctggtacgcaaagatctccagaatttcctgaagaaagaaaacaagaacgagaaagtcatcgaacacattatggaagatctggataccaacgcggataaacagttatcgtttgaggagttcattatgctgatggcacgtctgacatgggcctcacatgagaaaatgcatgaaggcgatgagggtcctggacatcatcacaaaccggggttaggcgaaggaactccgttggaagttctgtttcaaggcccacaccatcatcaccatcactaa

SEQ ID NO: 7 - protein, 229 aa, Artificial
  HisTag-3C Protease Seq.-MRP14-TEV Protease seq.[Linker]-MRP8
MHHHHHHLEVLFQGPMTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTPENLYFQSMLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 8 - protein, 229 aa, Artificial
  HisTag-3C Protease Seq.-MRP8-TEV Protease seq.[Linker]-MRP14
MHHHHHHLEVLFQGPMLTELEKALNSIIDVYHKYSLIKGNFHAVYRDDLKKLLETECPQYIRKKGADVWFKELDINTDGAVNFQEFLILVIKMGVAAHKKSHEESHKEENLYFQSMTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 9 - protein, 229 aa, Artificial
  HisTag-TEV Protease seq.-MRP14-3C Protease Seq. [Linker]-MRP8
MHHHHHHENLYFQSMTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTPLEVLFQGPMLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 10 - protein, 229 aa, Artificial
  HisTag-TEV Protease seq.-MRP8-3C Protease Seq. [Linker]-MRP14
MHHHHHHENLYFQSMLTELEKALNSIIDVYHKYSLIKGNFHAVYRDDLKKLLETECPQYIRKKGADVWFKELDINTDGAVNFQEFLILVIKMGVAAHKKSHEESHKELEVLFQGPMTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

SEQ ID NO: 11 - DNA, 282 bp, Homo sapiens
  misc_feature: Human S100A8/Mrp8 DNA sequence
atgttgaccgagctggagaaagccttgaactctatcatcgacgtctaccacaagtactccctgataaaggggaatttccatgccgtctacagggatgacctgaagaaattgctagagaccgagtgtcctcagtatatcaggaaaaagggtgcagacgtctggttcaaagagttggatatcaacactgatggtgcagttaacttccaggagttcctcattctggtgataaagatgggcgtggcagcccacaaaaaaagccatgaagaaagccacaaagagtag

SEQ ID NO: 12 - DNA, 282 bp, Homo sapiens; identical to SEQ ID NO: 11
  misc_feature: Human S100A9/Mrp14 DNA sequence

SEQ ID NO: 13 - protein, 8 aa, Artificial
  Recognition sequence for Rhinovirus 3C Protease (Prescission protease)
LEVLFQGP

SEQ ID NO: 14 - protein, 7 aa, Artificial
  Recognition sequence for TEV protease
ENLYFQG

SEQ ID NO: 15 - protein, 6 aa, Artificial
  Recognition sequence for thrombin
LVPRGS

SEQ ID NO: 16 - protein, 4 aa, Artificial
  Recognition sequence for factor Xa; X is E or D
  MISC_FEATURE (2)..(2): D or E
IXGR

SEQ ID NO: 17 - protein, 5 aa, Artificial sequence
  Recognition sequence for an enteropeptidase
DDDDK

SEQ ID NO: 18 - protein, 254 aa, Artificial
  HisTag-3C Protease Seq.-MRP14-3C Protease Seq.[Linker]-MRP8 (Isoform a)
MHHHHHHLEVLFQGPMTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTPLEVLFQGPMSLVSCLSEDLKVLFFRWGKSVGIMLTELEKALNSIIDVYHKYSLIKGNFHAVYRDDLKKLLETECPQYIRKKGADVWFKELDINTDGAVNFQEFLILVIKMGVAAHKKSHEESHKE

SEQ ID NO: 19 - protein, 254 aa, Artificial sequence
  HisTag-3C Protease Seq.- MRP8 (Isoform a)-3C Protease Seq.[Linker]- MRP14
MHHHHHHLEVLFQGPMSLVSCLSEDLKVLFFRWGKSVGIMLTELEKALNSIIDVYHKYSLIKGNFHAVYRDDLKKLLETECPQYIRKKGADVWFKELDINTDGAVNFQEFLILVIKMGVAAHKKSHEESHKELEVLFQGPMTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

(19) United States
(12) Patent Application Publication
(10) Pub. No.: US 2023/0074277 A1
(43) Pub. Date: Mar. 9, 2023

(54) Recombinant calprotectin

(71) Applicant: Buehlmann Laboratories AG, Schoenenbuch (CH)

(72) Inventors: Christian-Benedikt Gerhold, Efringen-Kirchen (DE); Michael Adrian Gerspach, Basel (CH); Dmitrii Guschin, Efringen-Kirchen (DE); Jakob Weber, Pfeffingen (CH); Michael Takacs, Loerrach (DE)

(21) Appl. No.: 17/801,135

(22) PCT Filed: Feb. 24, 2021
(86) PCT No.: PCT/EP2021/054605

§ 371 Date: Aug. 19, 2022

(30) Foreign Application Priority Data
Feb. 24, 2020 (EP) 20159115.3

(51) Int. Cl. C07K 14/47; G01N 33/68; C12N 15/63

(52) U.S. Cl. CPC C07K 14/4728; G01N 33/68; C12N 15/63; C07K 2319/20

(57) ABSTRACT

The present invention relates to polypeptides comprising comprising a first chain comprising an amino acid sequence having at least 80% sequence identity to SEQ ID NO:1; a second chain comprising an amino acid sequence having at least 80% sequence identity to SEQ ID NO:2; and a linker linking the first and second chain. In addition, the present invention is directed to methods of using these polypeptides as calibrators and standards in diagnostic methods.

### FIELD OF THE INVENTION

The present invention relates to polypeptides comprising a first chain comprising an amino acid sequence having at least 80% sequence identity to SEQ ID NO:1; a second chain comprising an amino acid sequence having at least 80% sequence identity to SEQ ID NO:2; and a linker linking the first and second chain. In addition, the present invention is directed to methods of using these polypeptides as calibrators and standards in diagnostic methods.

### BACKGROUND OF THE INVENTION

Calprotectin (CP) is a cytoplasmic protein expressed in various myeloid cell types, such as neutrophils, monocytes, and macrophages. In neutrophils, calprotectin is constitutively expressed and may constitute approximately 40% of the total cytoplasmic protein, while in epithelial cells and keratinocytes, calprotectin expression can be induced.

Calprotectin consists of the two polypeptide chains, Mrp8 (synonyms: S100A8, Calgranulin A) and Mrp14 (synonyms: S100A9, Calgranulin B), that form a stable dimer. In the presence of about 150 μM Ca²⁺, two Mrp8/Mrp14 heterodimers can form a heterotetramer, which has an important function in nutritional immunity as a sequestration complex for other divalent cations, such as Zn²⁺, to starve microbes during inflammation procedures (Zygiel EM, Nolan EM. Transition Metal Sequestration by the Host-Defense Protein Calprotectin (2018). Annu. Rev. Biochem. 87: 621-43).

Due to its release at inflammation sites, calprotectin is considered to be an alarmin and is frequently used as a biomarker to monitor inflammatory processes. For example, fecal calprotectin is currently the gold standard to diagnose and monitor inflammatory bowel diseases (IBD), such as Crohn's disease (CD) and Ulcerative Colitis (UC) (Konikoff M R, Denson L A. Role of Fecal Calprotectin as a Biomarker of Intestinal Inflammation in Inflammatory Bowel Disease (2006). Inflamm. Bowel Dis. 12 (6): 524-34).

Moreover, serum CP is validated as a biomarker to monitor various (chronic) inflammatory diseases, such as rheumatoid arthritis (Austermann J et al. S100 proteins in rheumatic diseases (2018). Nat. Rev. Rheumatol. 14: 528-541; Ometto F et al. Calprotectin in rheumatic diseases (2017). Exp. Biol. Med. 242: 859-873).

In order to develop reliable immunoassays to measure calprotectin, it is important to use a highly purified calprotectin antigen, which must be as similar as possible to native calprotectin. This avoids assay problems caused by differences in antibody reactivity between calibrators, controls and samples. Presently, calprotectin is mainly used as a fecal biomarker for distinguishing between organic IBD and non-organic irritable bowel syndrome (IBS), but there are no generally accepted and/or validated reference materials to be used to calibrate blood or fecal analytical assays.

Method comparisons show that there are clear calibration differences between fecal calprotectin assays from different manufacturers (De Sloovere M W et al. Analytical and Diagnostic Performance of Two Automated Fecal Calprotectin Immunoassays for Detection of Inflammatory Bowel Disease (2017). Clin. Chem. Lab. Med. 55 (9): 1435-1446), which severely aggravates test result interpretation by physicians.

State of the art methods for obtaining pure calprotectin include bacterial expression of Mrp8 and Mrp14 in insoluble inclusion bodies with elaborate refolding processes (Hadley and Nolan, Methods in Molecular Biology, 2019) or the purification of calprotectin from granulocytes isolated from human blood (Nilsen T, Haugen S H. Extraction, isolation, and concentration of calprotectin antigen (S100A8/S100A9) from granulocytes (2018). Health Sci. Rep. 1: e35).

However, both methods have several drawbacks. Refolding of Mrp8 and Mrp14 obtained from inclusion bodies may lead to homodimeric or other refolding artefacts (Vogl et al. Biophysical Characterization of S100A8 and S100A9 in the Absence and Presence of Bivalent Cations (2006). Biochim. Biophys. Acta 1763 (11): 1298-12306). On the other hand, purification of calprotectin from blood donors has a low yield, is labor-extensive and costly and does not allow for site directed mutagenesis of calprotectin.

There is therefore a need in the art for artificial calprotectin that is highly similar to natural calprotectin and can serve as a reliable calibrator and control for immunoassays and other analytical assays.

Objective Problem to be Solved

The problem to be solved is thus the provision of a recombinant calprotectin maintaining important characteristics of naturally occurring calprotectin.

### SUMMARY OF THE INVENTION

The problem is solved by a method for expressing soluble calprotectin, the method comprising expressing calprotectin from a vector comprising a first chain comprising a nucleotide sequence having at least 80% sequence homology to SEQ ID NO: 11, a second chain comprising a nucleotide sequence having at least 80% sequence homology to SEQ ID NO: 12 and a linker linking the first and second chain.

The problem is also solved by a polypeptide comprising
a) a first chain comprising an amino acid sequence having at least 80% sequence identity to SEQ ID NO:1;
b) a second chain comprising an amino acid sequence having at least 80% sequence identity to SEQ ID NO:2; and
c) a linker linking the first and the second chain,
   wherein the linker is a peptide linker comprising a protease recognition sequence.

The invention is also directed to a method for measuring S100A8, S100A9, S100A8/A9 or oligomers thereof in a sample using a polypeptide according to the invention in an analytical assay, comprising the steps of:
a) measuring different amounts of said polypeptide using the analytical assay;
b) establishing a calibration curve using the analytical results obtained in step a);
c) measuring a sample;
d) comparing the analytical result of the sample with the calibration curve of step b); and
e) quantifying the concentration of S100A8, S100A9, S100A8/A9 or oligomers thereof in the sample.

The invention is further directed to a polypeptide according to the invention for use in a method of diagnosing an acute or chronic inflammatory disease in a subject, the method comprising
a) providing a biological sample from the subject;
b) quantifying the amount of S100A8, S100A9, S100A8/A9 or oligomers thereof in the biological sample of step a) by using the polypeptide according to the invention oligomers thereof as calibration reference substance; and
c) comparing the amount of S100A8, S100A9, S100A8/A9 or oligomers thereof as determined in step b) to reference data from subjects known to suffer from an acute or chronic inflammatory disease.

The invention is also directed to a kit comprising
a) a polypeptide according to the invention and/or an oligomer thereof;
b) a test containment;
c) a buffer solution; and
d) a first binding reagent.

### BRIEF DESCRIPTION OF THE FIGURES

FIG. 1: Size exclusion chromatography of the fusion protein according to the invention in buffer A (20 mM HEPES pH 7.5, 100 mM NaCl, 2.5% glycerol, 1 mM DTT) on HiPrep Sephacryl S200 16/60 (GE Healthcare). The purified fusion protein is monodisperse and elutes as monomer (26.8 kDa) from the column.

FIG. 2: 3C digest of the fusion protein. 15 μg of purified rCAL (Mrp14-Mrp8 fusion) monomer was digested for 3 hrs at room temperature with 150 ng (++) or 50 ng (+) of purified 3C protease. Samples were then prepared for SDS-PAGE with LDS buffer (×4) and denatured at 95° C. for 12 minutes. SDS-PAGE revealed that the fusion protein is efficiently cleaved by 3C protease into S100A8 and S100A9 chains.

FIG. 3: The addition of 2 mM CaCl₂) to buffer A changes the elution volume of the recombinant fusion polypeptide of Example 1 significantly, which is indicative of dimerization. This is equivalent to the calcium dependent heterotetramerization of S100A8/S100A9 in endogenous calprotectin.

FIG. 4: Binding of the monoclonal antibody 27E10 to calprotectin requires heterodimerization of S100A8 and S100A9 (Hessian P A, Fisher L. The heterodimeric complex of MRP-8 (S100A8) and MRP-14 (S100A9). Antibody recognition, epitope definition and the implications for structure (2001). Eur. J. Biochem. 268: 353-363). The fusion protein displays high affinity to 27E10 mAB in BLI (biolayer interferometry) measurements, suggesting that the 3D structure of the fusion polypeptide according to the invention mimics the heterodimeric surface structure of the endogenous calprotectin heterotetramer.

FIG. 5: Sandwich ELISA measurement employing a dilution series of the recombinant fusion polypeptide (SEQ-ID NO:3) with monoclonal capture and detection antibodies. The capture antibody is linked to HRP which leads to a concentration dependent readout at OD450 after TMB addition. A linear correlation of spiked fusion polypeptide concentrations (from 0.5 μg/ml to 10 μg/ml) with the OD 450 signal suggests that the fusion polypeptide (SEQ-ID NO:3) can be recognized by monoclonal calprotectin antibodies in a concentration dependent manner.

FIG. 6: Turbidimetric measurement employing a dilution series of the recombinant fusion polypeptide (SEQ-ID NO:3) with polyclonal antibodies. The PETIA (particle enhanced turbidimetric immunoassay) also shows a correlating absorption signal with spiked fusion polypeptide concentrations (from Oto 21.7 μg/ml). Therefore, polyclonal antibodies directed against S100A8/S100A9 also readily recognize the fusion polypeptide (SEQ-ID NO:3) of the invention corroborating that its structure mimics endogenous calprotectin.

FIG. 7: Comparison of results obtained from 23 serum human samples with the turbidimetric immunoassay described in FIG. 6, whereby in one formulation the immunoassay was calibrated with purified endogenous calprotectin (S100A8/A9 heterotetramer) and in the second formulation with the recombinant fusion polypeptide (SEQ-ID NO:3) of the invention. The two result sets were then correlated and displayed as Passing-Bablok plot.

FIG. 8: Comparison of the results obtained from 4 different dilutions of the recombinant fusion polypeptide (SEQ-ID NO:3) of the invention and 23 serum human samples with the ELISA employing monoclonal antibodies as described in Example 5 (FIG. 5) versus the turbidimetric immunoassay employing polyclonal antibodies as described in Example 5 (FIG. 6). For this experiment, both assays were calibrated with endogenous calprotectin. The two result sets were then correlated and displayed as Passing-Bablok plot.

FIG. 9: Site-directed mutagenesis of the SEQ-ID NO:3 fusion protein and its consequences. A previously described mutation (EP3248015 A1) that impairs heterotetramerization of S100A8/S100A9, namely E78A in the S100A9 polypeptide chain, was introduced into the fusion protein. This E78A mutant remains monomeric, even in the presence of CaCl₂. S200 size exclusion chromatography in buffer A+2 mM CaCl₂ that shifts the non-mutated fusion protein to elution volumes indicating dimerization (at approx. 63 ml) does not alter the elution profile (at approx. 56 ml) of the fusion protein containing the E78A mutation when compared to buffer A without addition of calcium.

### DETAILED DESCRIPTION OF THE INVENTION

The present invention relates to a method for expressing soluble calprotectin, the method comprising expressing calprotectin from a vector comprising a first chain comprising a nucleotide sequence having at least 80% sequence homology to SEQ ID NO: 11, a second chain comprising a nucleotide sequence having at least 80% sequence homology to SEQ ID NO: 12 and a linker linking the first and second chain.

In preferred embodiments, the linker linking the first and the second chain on the vector has a length between 18 and 180 nucleotides, 18 and 120 nucleotides, 18 and 90 nucleotides or 18 and 60 nucleotides, most preferably between 21 and 30 nucleotides. Linkers having this length ensure sufficient proximity of the two chains to allow for correct folding.

As used herein, the term “S100A8” refers to a polypeptide having SEQ ID NO:1. Human S100A8, encoded by SEQ ID NO:11, is also known in the art to as Mrp8 or calgranulin A.

As used herein, the term “S100A9” refers to a polypeptide having SEQ ID NO:2. Human S100A9, encoded by SEQ ID NO:12, is also known in the art as Mrp14 or calgranulin B.

Heterodimers of S100A8 and S100A9 form immediately and spontaneously as soon as these two molecules come into contact with each other. In the presence of metal ions, in particular bivalent metal ions such as Ca²⁺, two S100A8/S100A9 heterodimers may form a heterotetramer. Herein, the term “oligomers of S100A8/S100A9” refers to heterodimers, heterotetramers and oligomers including multimers comprising one or more S100A8 and S100A9 monomers. In particular, the term “oligomers of S100A8/S100A9” refers to endogenous “calprotectin”, i.e. a heterotetramer comprising two units of each S100A8 and S100A9.

Prior to the invention, recombinant calprotectin was obtained by expressing each of the subunits separately into inclusion bodies, then purifying, refolding and dimerizing the subunits to form the naturally occurring heterodimers and heterotetramers. This process is not only labor intensive, but also error prone, since the procedure will often result in the formation of homodimers with little analytical value.

The inventors have surprisingly found that by linking the two subunits and having them expressed as one fusion protein, correctly folded heterodimers are obtained directly upon expression, thus greatly facilitating subsequent purification. Therefore, in a preferred embodiment, the methods of the invention do not comprise a step of extracellular refolding of the calprotectin. Put differently, the methods of invention ensure that the calprotectin is correctly folded already upon expression.

The heterodimers obtained by the methods of the invention subsequently assemble to heterotetramers in the presence of bivalent metal ions, similar to native calprotectin heterodimers. The soluble calprotectin obtained by the methods of the invention is structurally essentially identically to native calprotectin and is therefore capable of binding monoclonal antibodies that are specific for S100A8/S100A9 heterodimers, in particular monoclonal antibody mAb27E10.

Monoclonal antibodies having clone ID mAb27E10 can be obtained commercially, e.g. from Abcam, UK (ab17050) or Santa Cruz Biotechnology, Tex., USA (sc-33714). mAb27E10 has been further described in Hessian and Fisher, Eur. J. Biochem. 268, 353-363 (2001).

The present invention further relates to a polypeptide comprising
a) a first chain comprising an amino acid sequence having at least 80% sequence identity to SEQ ID NO:1;
b) a second chain comprising an amino acid sequence having at least 80% sequence identity to SEQ ID NO:2; and
c) a linker linking the first and second chain,
   wherein the linker is a peptide linker comprising a protease recognition sequence.

The polypeptides according to the invention comprise two chains comprising amino acid sequences having at least 80% sequence identity to SEQ ID NOs: 1 and 2, respectively. In the context of the invention, the term “sequences having at least 80% sequence identity” comprises sequences having at least 85%, at least 90%, at least 95%, at least 96%, at least 97%, at least 98%, at least 99%, at least 99.5%, most preferably 100% sequence identity to the respective sequence. In all cases, the identity is the identity over the total length of the corresponding amino acid sequence.

SEQ ID NO: 1 corresponds to human S100A8 and SEQ ID NO: 2 corresponds to human S100A9. Thus, the polypeptides according to the invention comprise amino acid sequences similar or identical to the two monomers constituting human calprotectin. Herein, it is envisaged that the first chain is one of the isoforms of S100A8 that are known in the art, in particular the isoform having 93 amino acids (SEQ ID NO:1). Likewise, the first chain may correspond to the S100A8 isoforms having 101, 116 or 117 amino acids.

The first chain of the polypeptides according to the invention is preferably at least 80, 85 or 90 amino acids longs. In a particularly preferred embodiment, the first chain is exactly 93, 101, 116 or 117 amino acids long.

The second chain of the polypeptides according to the invention is preferably at least 90, 100 or 110 amino acids longs. In a particularly preferred embodiment, the first chain is exactly 114 amino acids long.

In the polypeptides according to the invention, the two chains are connected by a a peptide linker. Such a linker has the advantage that it can be easily adapted and integrated into the polypeptide of the invention.

Preferably, the peptide linker is between 1 and 20 amino acids long. In a particularly preferred embodiment, the peptide linker is between 5 and 10 amino acids long. A peptide linker having this length is particularly suited for linking the two chains of the polypeptide according to the invention in a way that allows interaction between the two chains reminiscent of the dimer interactions of S100A8 with S100A9 in endogenous calprotectin.

The peptide linker comprises a protease recognition sequence, i.e. an amino acid sequence that can be recognized and cleaved by a protease. This has the advantage that the first and second chain of the polypeptide according to the invention can be separated from each other by addition of the respective protease. Thus, polypeptides according to this embodiment can be cut into the two chains corresponding to Mrp8 and Mrp14 once they have been expressed and assembled, thus allowing for restoration of the physiological state in which Mrp8 and Mrp14 are not linked permanently.

Many examples of protease recognition sequences are known in the art. For example, the protein recognition sequence may be LEVLFQGP for the Rhinovirus 3C Protease (Prescission protease), ENLYFQG for the TEV protease, LVPRGS for Thrombin, IEDGR for factor Xa or DDDDK for an Enteropeptidase.

The linker may link the first chain either N-terminally or C-terminally to the second chain. In a preferred embodiment, the first chain is linked N-terminally to the linker and the second chain. This arrangement is advantageous, since it facilitates soluble expression of the polypeptide in E. coli.

In one aspect of the invention, the polypeptides according to the invention are capable of oligomerizing, particularly dimerizing, with each other in the presence of metal ions. Preferably, the metal ions are divalent metal ions, most preferably, Ca²⁺. When oligomerizing, the polypetides of the invention are capable of forming a structure that is highly similar to naturally occurring calprotectin heterotetramers. This is useful for testing and calibrating immunoassays aiming at the detection of endogenous calprotectin.

In another aspect of the invention, the polypeptide according to the invention is capable of forming a structure that is recognized by a binding reagent recognizing the S100A8 monomer, S100A9 monomer, S100A8/S100A9 dimer and/or oligomers thereof, including endogenous calprotectin.

The binding reagent may be any entity recognizing the S100A8 monomer, S100A9 monomer, S100A8/S100A9 dimer and/or oligomers thereof, in particular calprotectin. In particular, the binding reagent may be an antibody or a fragment thereof, a sybody, a nanobody, a monobody, a CAMELID HC antibody, an affibody, a TandAb, a bicyclic petide, a DARPin, an avimer, an Ankyrin repeat sequence, a BiTE, a DART, an anticalin or a nucleotide sequence.

The polypeptide according to the invention may additionally comprise tags, markers, recognition sequences or signals known in the art to allow for purification, identification, localization and recognition of the polypeptides according to the invention. In one embodiment, the polypeptide according to the invention comprises a His-Tag allowing for efficient purification of the polypeptide using chromatography. Other tags or markers that may be used in the polypeptide according to the invention are FLAG-Tag, HA-Tag, Strep-Tag or GFP which are all known to the skilled person.

In a preferred embodiment, the polypeptide according to the invention has at least 80% sequence identity to SEQ ID NO: 7, 8, 9, 10, 18 or 19. In a preferred embodiment, the polypeptide according to the invention has 85%, 90% or 95% sequence identity to SEQ ID NO: 7, 8, 9, 10, 18 or 19. In a particularly preferred embodiment, the polypeptide according to the invention has 100% sequence identity to SEQ ID NO: 7, 8, 9, 10, 18 or 19. SEQ ID NO: 7 to 10 comprises a His-Tag linked to the first or second chain by a short amino acid sequence comprising the protease sequence for Rhinovirus SC Protease so that the His-Tag can be cleaved from the part of the polypeptide comprising the two chains and the linker by addition of Rhinovirus 3C Protease. Thus, a polypeptide having SEQ ID NO: 7 to 10 can be effectively purified and the His-Tag be subsequently disposed of. In addition, the linker between the two chains used in SEQ ID NO: 7 to 10 comprises the recognition sequence for the TEV protease, so that the two chains can be disassociated by addition of the TEV protease.

In other embodiments, shown in SEQ ID NO: 3 and 4, the polypeptides according to the invention comprise the same protease recognition sequence between the His-Tag and the two chains, thus allowing for removal of the His-Tag and cleavage between of two chains in a single step.

In further embodiments, the chains of the polypeptides according to the invention carry one or more specific mutations compared to naturally occurring S100A8 and S100A9. The mutations may affect oligomerization of the chains and/or recognition by specific binding agents.

For example, the polypeptides according to the invention may carry mutations such as the ones described in EP3248015 A1, which prevent oligomerization between heterodimers of S100A8 and S100A9. These polypeptides may be advantageously used as calibrators or standards since they provide a well-defined structure that is not influenced by external conditions. For example, they do not heterotetramerize in presence of calcium ions and/or other substances either present in the assay buffer or in a biological sample.

The present invention is also directed to polynucleotide sequences encoding the polypeptides according to the invention. In one embodiment, the polynucleotide sequence is a polynucleotide sequence that hybridizes under high stringency conditions with the polynucleotide sequence of SEQ ID NO: 5. In another embodiment, the polynucleotide sequence is a polynucleotide sequence that hybridizes under high stringency conditions with the polynucleotide sequence of SEQ ID NO: 6. SEQ ID NO: 5 encodes for the amino acid sequence of SEQ ID NO: 3, while SEQ ID NO: 6 encodes for the amino acid sequence of SEQ ID NO: 4.

The term “hybridization” or “hybridize” as used herein includes any process by which a strand of nucleic acid molecule joins with a complementary strand through base pairing” (J. Coombs (1994) Dictionary of Biotechnology, Stockton Press, New York). Hybridization and the strength of hybridization (i.e., the strength of the association between the nucleic acid molecules) is impacted by such factors as the degree of complementarity between the nucleic acid molecules, stringency of the conditions involved, the Tm of the formed hybrid, and the G:C ratio within the nucleic acid molecules.

As used herein, the term “Tm” is used in reference to the “melting temperature”. The melting temperature is the temperature at which a population of double-stranded nucleic acid molecules becomes half dissociated into single strands. The equation for calculating the Tm of nucleic acid molecules is well known in the art. As indicated by standard references, a simple estimate of the Tm value may be calculated by the equation: Tm=81.5+0.41 (% G+C), when a nucleic acid molecule is in aqueous solution at 1 M NaCl (see e.g., Anderson and Young, Quantitative Filter Hybridization, in Nucleic Acid Hybridization (1985)). Other references include more sophisticated computations, which take structural as well as sequence characteristics into account for the calculation of Tm. Stringent conditions, are known to those skilled in the art and can be found in Current Protocols in Molecular Biology, John Wiley & Sons, N.Y. (1989), 6.3.1-6.3.6.

In particular, the term “stringency conditions” refers to conditions, wherein 100 contigous nucleotides or more, 150 contigous nucleotides or more, 200 contigous nucleotides, 250 contigous nucleotides or more which are a fragment or identical to the complementary nucleic acid molecule (DNA, RNA, ssDNA or ssRNA) hybridizes under conditions equivalent to hybridization in 7% sodium dodecyl sulfate (SDS), 0.5 M NaPO4, 1 mM EDTA at 50° C. with washing in 2×SSC, 0.1% SDS at 50° C. or 65° C., preferably at 65° C., with a specific nucleic acid molecule (DNA; RNA, ssDNA or ss RNA). Preferably, the hybridizing conditions are equivalent to hybridization in 7% sodium dodecyl sulfate (SDS), 0.5 M NaPO4, 1 mM EDTA at 50° C. with washing in 1×SSC, 0.1% SDS at 50° C. or 65° C., preferably 65° C., more preferably the hybridizing conditions are equivalent to hybridization in 7% sodium dodecyl sulfate (SDS), 0.5 M NaPO4, 1 mM EDTA at 50° C. with washing in 0.1×SSC, 0.1% SDS at 50° C. or 65° C., preferably 65° C. Preferably, the complementary nucleotides hybridize with a fragment or the whole nucleic acids. Alternatively, preferred hybridization conditions encompass hybridisation at 65° C. in 1×SSC or at 42° C. in 1×SSC and 50% formamide, followed by washing at 65° C. in 0.3×SSC or hybridisation at 50° C. in 4×SSC or at 40° C. in 6×SSC and 50% formamide, followed by washing at 50° C. in 2×SSC. Further preferred hybridization conditions are 0.1% SDS, 0.1 SSD and 65° C.

The polynucleotide sequences according to the invention may additionally carry promoters, nuclease recognition sites, marker genes, enhancers and other genetic elements useful for transformation with and expression of polynucleotide sequences.

The polypeptides according to the invention may form oligomers. Therefore, in one embodiment, the invention is directed to polypeptide oligomers comprising two or more polypeptides according to the invention. In a preferred embodiment, the polypeptide oligomer comprises exactly two polypeptides according to the invention. Such a polypeptide oligomer corresponds to endogenous calprotectin heterotetramers comprising two S100A8 and two S100A9 monomers. In a particularly preferred embodiment, the polypeptide oligomers are formed in the presence of bivalent metal ions, preferably Ca²⁺.

The polypeptides according to the invention or oligomers thereof may be used for the immunization of an animal. “Immunization” herein refers to the administration of a substance to a subject or an animal with the aim of inducing an immune response in the subject or animal. The immune response may involve the generation of antibodies.

The animal may be any animal useful for clinical research or industrial applications, e.g. a mouse, a rat, a rabbit, a goat, a dog, a hen, a shark, a camelid, a pig or a monkey. When immunizing an animal with the polypeptides according to the invention, the polypeptides may be formulated in any way known in the art, for example by addition of further immunogenic compounds.

In the process of immunization, the polypeptides according to the invention can replace endogenous calprotectin for obtaining antibodies directed against calprotectin.

The polypeptides according to the invention or oligomers thereof may also be used as epitope for in vitro selection or for the affinity purification of a binding reagent recognizing the S100A8 monomer, S100A9 monomer, S100A8/S100A9 dimer and/or oligomers thereof, in particular endogenous calprotectin. Since polypeptides according to the invention can be easily obtained in large quantities, using them in in vitro selection and purification methods allows for cost-effective selection and purification of suitable binding reagents in a reproducible manner.

In another embodiment, the polypeptides according to the invention may be used as a medicament. In one embodiment, the medicament may be useful for treating or preventing diseases that require modulation of the immune response or reduction/inhibition of inflammatory processes.

In another preferred embodiment, the polypeptides according to the invention may be used as a calibration reference substance. A calibration reference substance is herein understood as referring to a substance used either as (the internationally recognized) primary reference material (of highest order) for the traceability and/or comparability of any kind of analytical assays or as a standard when establishing a novel analytical assay. Thus, a calibration reference substance can be used to test sensitivity and specificity of novel binding agents or assay systems. Likewise, a calibration reference substance can be used for calibrating an analytical assay. The polypeptides according to the invention can therefore be used in a method of establishing novel assays, in particular, immunoassays, and for the testing of binding agents as well as for calibrating an assay. In these embodiments, the polypeptides according to the invention can replace standard heterodimeric or heterotetrameric calprotectin as calibration reference substance because the polypeptides according to the invention show all essential characteristics of native calprotectin such as epitope presentation and binding properties, but are easier to obtain than calprotectin previously used as calibration reference substance.

The invention is also directed to a method for measuring S100A8, S100A9, S100A8/A9 or oligomers thereof, in particular calprotectin, in a sample using a polypeptide according to the invention in an analytical assay, comprising the steps of:
a) measuring different amounts of said polypeptide using the analytical assay;
b) establishing a calibration curve using the analytical results obtained in step a);
c) measuring a sample;
d) comparing the analytical result of the sample with the calibration curve of step b); and
e) quantifying the concentration of S100A8, S100A9, S100A8/A9 or oligomers thereof in the sample.

The person skilled in the art knows how to calibrate an analytical assay. Briefly, different amounts of the polypeptide according to the invention are measured using the analytical assay to obtain analytical results that are then used to establish a calibration curve. In other words, polypeptides according to the invention or oligomers thereof are used as calibration reference substance for the analytical assay. Because of their monodispersity and homogeneity, calibration using the polypeptides according to the invention is fast and highly reliable.

In the method according to the invention, the sample is then measured using the same analytical test and the analytical results of these measurements are compared to the calibration curve. From the comparison, the concentration of S100A8, S100A9, S100A8/A9 or oligomers thereof in the sample can be quantified.

The polypeptide according to the inventions or oligomers thereof can also be used in a method of diagnosing an acute or chronic inflammatory disease in a subject, the method comprising
a) providing a biological sample from the subject;
b) quantifying the amount of S100A8, S100A9, S100A8/A9 or oligomers thereof, in particular calprotectin, in the biological sample of step a) by using the polypeptide according to the invention oligomers thereof as calibration reference substance; and
c) comparing the amount of S100A8, S100A9, S100A8/A9 or oligomers thereof as determined in step b) to reference data from subjects known to suffer from an acute or chronic inflammatory disease.

The person skilled in the art knows various types of acute or chronic inflammatory diseases, e.g. allergy, asthma, autoimmune diseases, coeliac disease, glomerulonephritis, hepatitis, inflammatory bowel disease, Ulcerative Colitis, Crohn's disease, preperfusion injury, transplant rejection, infectious colitis, necrotizing enterocolitis, (intestinal) cystic fibrosis, rheumatoid arthritis, juvenile idiopathic arthritis, ankylosing spondylitis, swollen joints, psoriasis, psoriatric arthritis, Behcet disease, gingivitis, tonsillitis, appendicitis, lupus, fever of unknown origin, sepsis, cardiac diseases, myocardial infarction, multiple sclerosis, and cancer such as colorectal cancer. In a preferred embodiment, the inflammatory disease is selected from the group consisting of inflammatory bowel disease, in particular Ulcerative Colitis or Crohn's disease, and inflammatory arthritis, in particular rheumatoid arthritis, juvenile idiopathic arthritis or ankylosing spondylitis.

In one aspect of the invention, a significantly increased amount of S100A8, S100A9, S100A8/A9 or oligomers thereof, in particular calprotectin, as compared to reference data indicates that a subject suffers from an acute or chronic inflammatory disease.

Using the polypeptides according to the invention or oligomers thereof, inflammatory diseases can be easily and reliably diagnosed and their therapies monitored.

In the methods according to the invention, the sample may be any biological sample used in the art, in particular blood, serum, plasma, synovial fluid, saliva, urine, tears, sweat, gingival crevicular fluid, feces, gastrointestinal lavage, bronchial lavage, cell culture supernatant or tissue extract. In a preferred embodiment, the sample is a feces sample or a blood sample.

The analytical assay may be any analytical assay known in the art, e.g., an immunoassay, a biochemical assay, a biophysical assay or a physical assay. In a preferred embodiment, the analytical assay is an immunoassay based on methods that require recognition of the analyte by high affinity binding reagents, such as enzyme-linked immune absorbent assays (ELISA), lateral flow immunoassay (LFIA) or particle enhanced immunoturbidimetric assays (PETIA).

In the methods according to the invention, the analytical result of the sample may be based on an optical readout, absorption, UV/VIS spectroscopy, light scattering, turbidimetry, nephelometry, light scattering, reflectometry, fluorescence, luminescence, chemiluminescense, surface plasmon resonance, amperometry, magnetometry, voltametry, potentiometry, conductometry, coulometry, polarography, gravimetry or cantilevers. In a preferred embodiment, the analytical result is based on an immunoassay measured by absorption, UV/VIS spectroscopy, light scattering, turbidimetry, nephelometry, reflectometry, fluorescence, luminescence or chemiluminescense. In the most preferred embodiment the analytical result is based on an immunoassay measured by absorption spectroscopy, light scattering or chemiluminescense.

The invention is also directed to a kit comprising
a) a polypeptide according to the invention and/or an oligomer thereof;
b) a test containment;
c) a buffer solution;
d) a first binding reagent, preferably immobilized on a solid support, specific for S100A8, S100A9, S100A8/A9 or oligomers thereof, in particular calprotectin;

In one embodiment the first binding reagent is labeled with a substance or bound to a material allowing a quantitative determination of said polypeptide and S100A8, S100A9, S100A8/A9 or oligomers thereof.

The test containment is used for performing an analytical assay therein. For example, the test containment may be a test cartridge, a membrane, a tube, a titer plate or a vessel.

The person skilled in the art knows how to choose a suitable buffer solution. For example, the buffer solution may be phosphate, maleate, chloroacetate, formate, benzoate, pyridine, piperazine, propionate, 3-N-morpholinopropanesulfonic acid (MOPS), 1,3-bis tris-hydroxymethyl) methylaminopropane (Bis-TRIS), tris-(hydroxymethyl) aminomethane (TRIS), tris-(hydroxymehtyl) aminomethane-maleic acid (TRIS-maleate), 2-(-tris-(hydroxymethyl)methylamino)ethanesulfonic acid (TES), 1,4-piperazinebis-ethanesulfonic acid) (PIPES), 4-morpholinoethanesulfonic acid (MES), N-2-hydroxyethylpiperazine-N′-2-ethanesulfonic acid (HEPES), N,N-bis(2-hydroxyethyl)-2-aminoethanesulfonic acid (BES), N-(2-acetamido)iminodiacetic acid (ADA), N-(2-acetamido)-2-aminoethanesulfonic acid (ACES), and others known to a person skilled in the art. The buffered solution may additionally comprise salts, antimicrobial agents, detergents, chelating agents, chaotropic agents, and/or anti-foaming agents.

The kit according to the invention further comprises a first and, optionally, a second binding agent that is/are specific for S100A8, S100A9, S100A8/S100A9 or oligomers thereof. In a preferred embodiment, the first/second binding agent is specific for heterotetramers comprising S100A8 and S100A9, i.e. calprotectin. In another preferred embodiment, the first/second binding agent is specific for the heterodimer comprising S100A8 and S100A9. It is, however, also envisaged by the invention that the first/second binding agent is specific for S100A8 or S100A9 monomers.

Because of the sequence of the polypeptides according to the invention, binding agents that are specific for S100A8, S100A9, S100A8/S100A9 or oligomers thereof also recognize the polypeptides according to the invention.

In a preferred embodiment, the first binding agent is immobilized on a solid support. This allows for the kit to be used in an ELISA, LFIA or PETIA.

According to the invention, the first/second binding reagent may be labeled with a substance or bound to a material allowing a quantitative determination of said polypeptides and S100A8, S100A9, S100A8/A9 or oligomers thereof. The person skilled in the art knows which substances or labels can be used for the quantitative determination of an analyte. For example, the binding agent may be labeled with latex, fluorescent, (para)magnetic, colored cellulose, ferric, gold or silica (nano)particles; fluorophores or fluorescent dyes; quantum dots; enzymes such as horse radish peroxidase (HRP), alkaline phosphates (AP), glucose oxidase, glucose-6-phosphate dehydrogenase, malate dehydrogenase, NADH dehydrogenase, acytelcholinesterase; chemiluminescent reactants such as luciferase or luminol and derivatives; fluorescent proteins such green fluorescent protein (GFP), red fluorescent protein or yellow fluorescent protein; and radioisotopes.

Optionally, the kit may comprise means to purify a biological sample.

The kit according to the invention thus allows quantitative determination of calprotectin in a sample. Because the polypeptides according to the invention specifically form heterodimers or heterotetramers, their use in the kit avoids false positives and thus allows for a reliable quantification of calprotectin in a sample.

### EXAMPLES

### Example 1: Preparation of a Polypeptide of the Present Invention

SEQ ID NO:5 was cloned into the pET21 vector (Novagen) for IPTG inducible T7 transcription. The expression strain E. coli BL21 was transformed with the corresponding plasmid. Induction of protein expression by IPTG was performed according to standard protocols to obtain soluble polypeptide having SEQ-ID NO:3. More specifically, the respective expression strain was grown to OD600=0.5 at 37° C., 200 rpm in LB medium with 100 μg/ml Carbenicillin. Subsequently, the cell culture was chilled to 18° C. and induction of protein expression was started by addition of 0.2 mM IPTG at OD600≈0.8. Expression was carried out at 18° C., 200 rpm for 14-16 hours before the E. coli expression strain was harvested by centrifugation at 7,000×g and washed in buffer A (20 mM HEPES pH 7.5, 100 mM NaCl, 2.5% glycerol, 1 mM DTT).

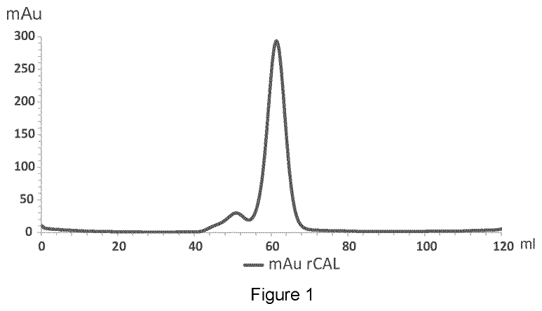

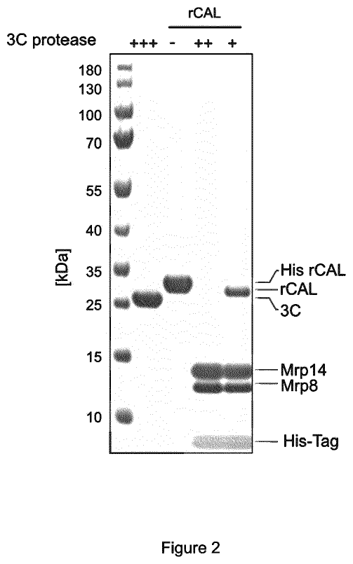

Resuspension of the induced expression strain in Lysis buffer (buffer A+20 mM imidazole) and subsequent cell disruption by sonication (Branson Sonifier) allowed separation of soluble proteins from cell debris by centrifugation at 20,000×g. The supernatant was applied to NiNTA resin and the purified fusion protein was eluted with an imidazole gradient. Application to a Q Sepharose column separated the fusion protein from nucleic acid contaminants using a NaCl gradient. A final purification step using an S200 size exclusion column (FIG. 1) gave rise to pure and monodisperse fusion protein in buffer A (FIG. 2, lane 3).

### Example 2: Proteolytic Cleavage of the Fusion Polypetide into its Monomers

Due to the 3C protease (PreScission™) recognition sequence linking the His-Tag with S100A9 as well as S100A9 with S100A8 in this version of the fusion protein (SEQ ID NO:3), the single fusion polypeptide can be proteolytically cleaved into three polypeptides, which correspond to the free His-Tag, S100A8 and S100A9 with the respective residual amino acids from the 3C recognition sequence (FIG. 2, lane 4).

### Example 3: Dimerization of the Recombinant Fusion Polypeptide in the Presence of Ca²⁺ Ions

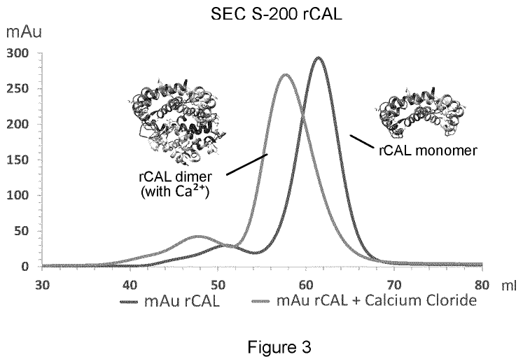

The recombinant fusion polypeptide shares characteristics with endogenous calprotectin, which is a heterodimer of S100A8 and S100A9. Size exclusion chromatography in buffer A containing 2 mM CaCl₂ gave a significant shift in the elution volume of the fusion protein indicating dimerization, which is equivalent to calcium dependent heterotetramerization of two S100A8 and S100A9 heterodimers (FIG. 3). The recombinant polypeptide monomer (equal to a S100A8/S100A9 dimer) elutes at approx. 62 ml, whereas the recombinant polypeptide dimer (equal to a S100A8/A9 tetramer) elutes more rapid at approx. 58 ml.

### Example 4: Immunogenic Properties of the Recombinant Fusion Polypeptide According to Invention

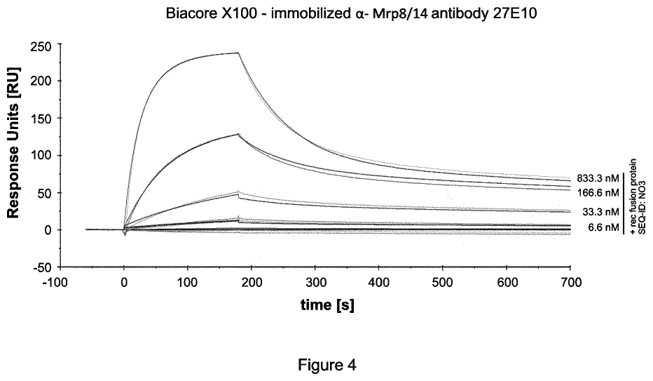

The described fusion protein (SEQ ID NO:3) displays immunogenic properties similarly to endogenous calprotectin. The monoclonal antibody 27E10 that only binds the dimer of S100A8 and S100A9, but not the individual monomers (Hessian P A, Fisher L. The heterodimeric complex of MRP-8 (S100A8) and MRP-14 (S100A9). Antibody recognition, epitope definition and the implications for structure (2001). Eur. J. Biochem. 268: 353-363). The monoclonal antibody 27E10 was immobilized on a Biacore chip sensor and recognized the fusion protein (SEQ ID NO:3), which is indicated by concentration dependent response units upon interaction with the fusion protein (SEQ ID NO:3) using surface plasmon resonance (FIG. 4).

### Example 5: Immunoassays Based on the Recombinant Fusion Polypeptide According to Invention

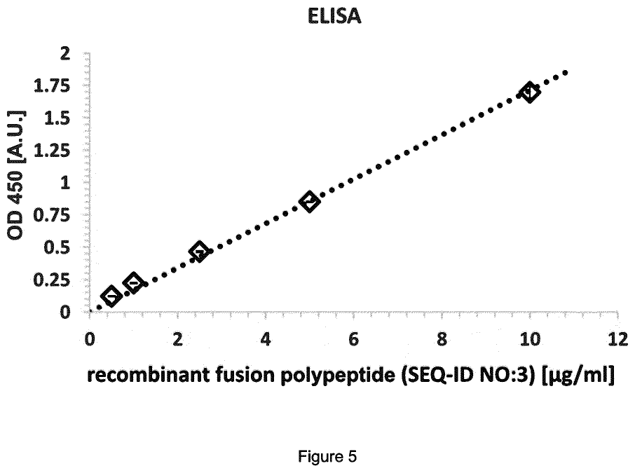

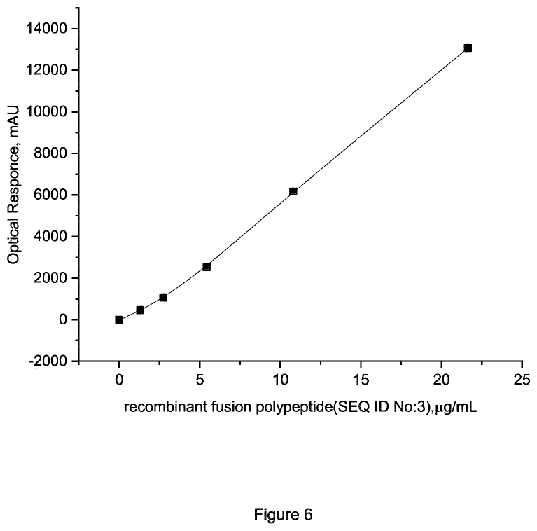

An ELISA with a monoclonal capture antibody was performed with different concentrations of the fusion protein (SEQ ID NO:3) in presence of CaCl₂). An HRP conjugated detection antibody was used to induce TMB (3,3′,5,5′-Tetramethylbenzidine) based color change. After addition of an acidic stop solution, the OD at 450 nm was measured and showed a linear correlation compared with spiked concentrations of purified fusion protein (SEQ-ID NO:3) that were previously assessed by A280 absorption measurements (FIG. 5). Similarly, an immunoturbidimetric test with polyclonal antibodies also correlates with different concentrations of the fusion protein (SEQ ID NO:3) (FIG. 6) corroborating that the fusion protein (SEQ ID NO:3) displays essentially the same characteristics as described for endogenous calprotectin (S100A8/S100A9), and thus can be used interchangeably with S100A8/S100A9 purified from endogenous human sources such as stool samples or blood derived granulocytes.

### Example 6: Interchangeability of Calibrators Consisting of Either Endogenous Calprotectin or Recombinant Fusion Polypeptide According to the Invention

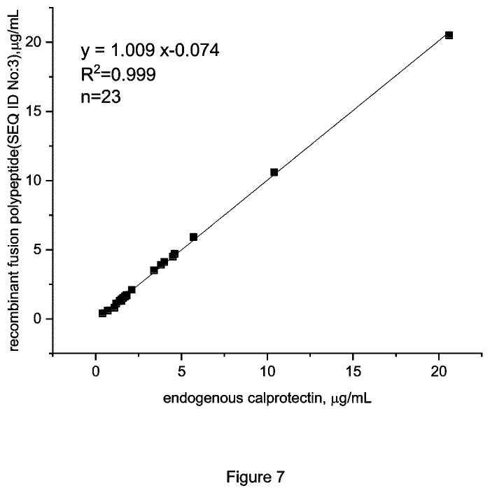

Turbidimetric determination of 23 human serum samples using the recombinant fusion polypeptide (SEQ ID NO:3) of the invention as calibrators and comparison with the results obtained the same immunoassay employing purified endogenous calprotectin (S100A8/A9 heterotetramer) as calibrator material. The results were plotted against each other and revealed a perfect correlation (slope=1.009; R²=0.999) independently of the calibrator material used (FIG. 7).

### Example 7: The Fusion Polypeptide of the Invention Shows the Same Immunological Properties Towards Polyclonal and Monoclonal Antibodies

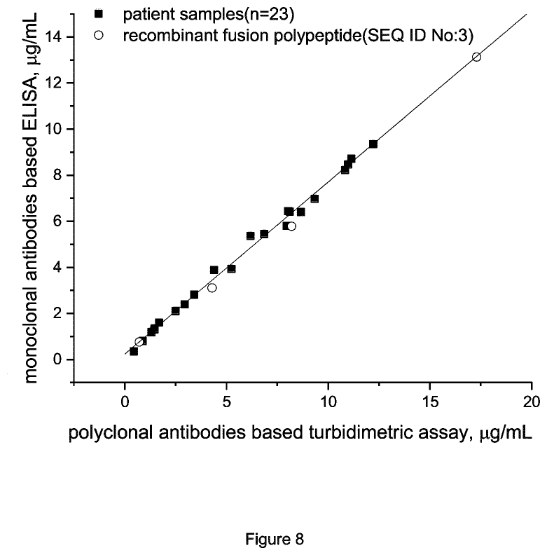

Four different dilutions of the recombinant fusion polypeptide as well as 23 serum human samples were analyzed with the ELISA employing monoclonal antibodies as described in Example 5 versus the turbidimetric immunoassay employing polyclonal antibodies also described in Example 5. For this experiment, both assays used endogenous calprotectin as calibration material. The two result sets were correlated and displayed as Passing-Bablok plot (FIG. 8). There was virtually no quantitative difference observed between the results generated by the polyclonal versus the monoclonal antibodies, neither for the fusion polypeptide (SEQ ID NO:3) of the invention nor for the human serum samples.

### Example 8: Prevention of Di- or Oligomerization of the Fusion Polypeptide (SEQ-ID NO:3) of the Invention by Introducing a Point Mutation

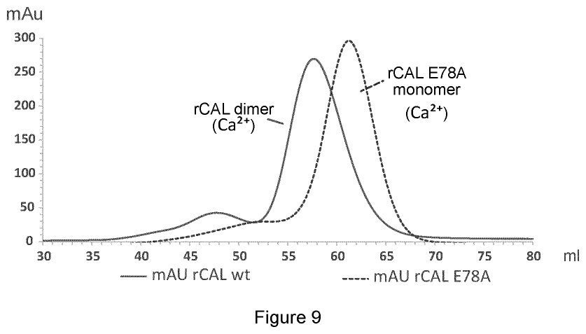

Site directed mutagenesis was used to introduce the mutation E78A in the S100A9 chain (see EP3248015 A1) of the fusion protein (SEQ ID NO:3). In endogenous calprotectin, this mutation abrogates heterodimerization (Leukert N et al. Calcium-dependent tetramer formation of S100A8 and S100A9 is essential for biological activity (2006). J. Mol. Biol. 359: 961-972). Similary, introduction of this mutation into the fusion protein (SEQ-ID NO:3) prevented CaCl₂) dependent dimerization. The S200 size exclusion chromatography of the mutated and non-mutated fusion proteins (SEQ ID NO:3) in buffer A containing 2 mM CaCl₂) was performed as described in Examples 1 and 3 (FIG. 9).

1. A method for obtaining soluble calprotectin, the method comprising expressing calprotectin from a vector comprising a first chain comprising a nucleotide sequence having at least 80% sequence homology to SEQ ID NO: 11, a second chain comprising a nucleotide sequence having at least 80% sequence homology to SEQ ID NO: 12 and a linker linking the first and second chain.

2. The method according to claim 1, wherein the soluble calprotectin is capable of binding monoclonal antibody mAb27E10.

3. The method according to claim 1 or 2, wherein the method does not comprise a step of extracellular refolding of the calprotectin.

4. The method according to any of claims 1 to 3, wherein the linker has a length between 18 and 180 nucleotides, preferably between 21 and 30 nucleotides.

5. The method according to any of claims 1 to 4, wherein the linker comprises a nucleotide sequence encoding a protease recognition sequence, in particular a Rhinovirus 3C Protease recognition sequence, a TEV protease recognition sequence, a thrombin recognition sequence, a factor Xa recognition sequence or an enteropeptidase recognition sequence.

6. A polypeptide comprising
a) a first chain comprising an amino acid sequence having at least 80% sequence identity to SEQ ID NO:1;
b) a second chain comprising an amino acid sequence having at least 80% sequence identity to SEQ ID NO:2; and
c) a linker linking the first and second chain,
wherein the linker is a peptide linker comprising a protease recognition sequence.

7. The polypeptide according to claim 4, wherein the protease recognition sequence is a Rhinovirus 3C Protease recognition sequence, a TEV protease recognition sequence, a thrombin recognition sequence, a factor Xa recognition sequence or an enteropeptidase recognition sequence.

8. The polypeptide according to claim 4 or 5, wherein the polypeptide is capable of oligomerizing in the presence of metal ions.

9. The polypeptide according to claim 6, wherein the polypeptide is capable of forming a dimer in the presence of divalent metal ions, preferably Ca²⁺ ions.

10. The polypeptide according to any of claims 4 to 7, wherein the polypeptide is capable of forming a structure that is recognized by a binding reagent recognizing the S100A8 monomer, S100A9 monomer, S100A8/S100A9 dimer and/or oligomers thereof.

11. The polypeptide according to claim 4 comprising an amino acid sequence having at least 80% sequence identity to the polypeptide of SEQ ID NO:3.

12. The polypeptide according to claim 4 comprising an amino acid sequence having at least 80% sequence identity to the polypeptide of SEQ ID NO:4, 7 to 10, 18 or 19.

13. A polypeptide oligomer comprising two or more polypeptides according to any of claims 6 to 12.

14. Use of a polypeptide according to any of claims 6 to 12 or obtained by the method according to any of claims 1 to 5 or a polypeptide oligomer according to claim 13 for the immunization of an animal.

15. Use of a polypeptide according to any of claims 6 to 12 or obtained by the method according to any of claims 1 to 5 or a polypeptide oligomer according to claim 13 as an epitope for the in vitro selection of a binding reagent recognizing the S100A8 monomer, S100A9 monomer, S100A8/S100A9 dimer and/or oligomers thereof.

16. Use of a polypeptide according to any of claims 6 to 12 or obtained by the method according to any of claims 1 to 5 or a polypeptide oligomer according to claim 13 for the affinity purification of a binding reagent recognizing the S100A8 monomer, S100A9 monomer, S100A8/S100A9 dimer and/or oligomers thereof.

17. A polypeptide according to claims 6 to 12 or obtained by the method according to any of claims 1 to 5 or a polypeptide oligomer according to claim 13 for use as a medicament.

18. Use of a polypeptide according to any of claims 6 to 12 or obtained by the method according to any of claims 1 to 5 or a polypeptide oligomer according to claim 13 as a calibration reference substance.

19. Method for measuring S100A8, S100A9, S100A8/A9 dimers or oligomers thereof in a sample using a polypeptide according to any of the preceding claims in an analytical assay, comprising the steps of:
a) measuring different amounts of said polypeptide using the analytical assay;
b) establishing a calibration curve using the analytical results obtained in step a);
c) measuring a sample using the analytical assay;
d) comparing the analytical result of the sample with the calibration curve of step b); and
e) quantifying the concentration of S100A8, S100A9, S100A8/A9 dimers or oligomers thereof in the sample.

20. Method according to claim 19, wherein the sample is blood, serum, plasma, synovial fluid, saliva, urine, tears, sweat, gingival crevicular fluid, feces, gastrointestinal lavage, bronchial lavage, cell culture supernatant or tissue extract.

21. Method according to claim 19 or 20, wherein the analytical assay is an immunoassay, a biochemical assay, a biophysical assay or a physical assay.

22. Method according to any of claims 19 to 21, wherein the anayltical result of the sample is based on an optical readout, absorption, UV/VIS spectroscopy, turbidimetry, nephelometry, light scattering, reflectometry, fluorescence, luminescence, chemiluminescense, surface plasmon resonance, amperometry, magnetometry, voltametry, potentiometry, conductometry, coulometry, polarography, gravimetry or cantilevers.

23. A polypeptide according to any of claims 6 to 12 or obtained by the method according to any of claims 1 to 5 or a polypeptide oligomer according to claim 13 for use in a method of diagnosing an acute or chronic inflammatory disease in a subject, comprising
a) providing a biological sample from the subject;
b) quantifying the amount of S100A8, S100A9, S100A8/A9 dimers or oligomers thereof in the biological sample of step a) by using a polypeptide according to any of claims 6 to 12 or obtained by the method according to any of claims 1 to 5 or a polypeptide oligomer according to claim 13 as calibration reference substance; and
c) comparing the amount of S100A8, S100A9, S100A8/A9 dimers or oligomers thereof as determined in step b) to reference data from subjects known to suffer from an acute or chronic inflammatory disease.

24. Kit comprising
a) a polypeptide according to any of claims 6 to 12 or obtained by the method according to any of claims 1 to 5 or a polypeptide oligomer according to claim 13
b) a test containment;
c) a buffer solution; and
d) a first binding reagent, specific for S100A8, S100A9, S100A8/A9 dimers or oligomers thereof.

25. Kit according to claim 24, wherein the first binding reagent is labeled with a substance or bound to a material allowing a quantitative determination of said polypeptides and S100A8, S100A9, S100A8/A9 dimers or oligomers thereof.

26. Kit according to claim 25, additionally comprising a second binding reagent specific for S100A8, S100A9, S100A8/A9 dimers or oligomers thereof, wherein the first binding reagent is immobilized on a solid support and the second binding reagent is labeled with a substance or bound to a material allowing a quantitative determination of said polypeptides and S100A8, S100A9, S100A8/A9 dimers or oligomers thereof.
